(19) United States
(12) Patent Application Publication
(10) Pub. No.: US 2005/0203333 A1
(43) Pub. Date: Sep. 15, 2005

(54) Magnetized scleral buckle, polymerizing magnetic polymers, and other magnetic manipulations in living tissue

(76) Inventors: James P. Dailey, Erie, PA (US); Judy Riffle, Blacksburg, VA (US)

Correspondence Address: Whitham, Curtis & Christofferson, PC, 11491 Sunset Hills Road, Suite 340, Reston, VA, 20190, US

(21) Appl. No.: 11/010,567

(22) Filed: Dec. 14, 2004

Related U.S. Application Data
(60) Provisional application No. 60/529,416, filed on Dec. 15, 2003.

(51) Int. Cl. A61F 2/14; A61F 9/007

(52) U.S. Cl. 600/37; 128/898; 623/4.1; 600/12

(57) ABSTRACT

A magnetic polymer may be polymerized in living tissue. Retinal detachment may now be repaired without needing suturing, by using a magnetic fluid with a magnetic scleral buckle. The magnetic scleral buckle may be polymerized into place in the eye, rather than being preformed outside the eye as has been conventionally done. Magnetic systems formed internally may be used in other medical contexts, such as in drug delivery.

Priority is claimed based on U.S. provisional application No. 60/529,416 filed Dec. 15, 2004, titled, “Magnetized Scleral Buckle.”

### FIELD OF THE INVENTION

The present invention is directed to biocompatible magnetic systems and medical procedures, especially repair of retinal detachment.

### BACKGROUND OF THE INVENTION

Retinal Detachment is a leading cause of blindness. Conventional treatments fail in as many as ⅓ of complicated retinal detachment patients, resulting in partial or complete loss of vision for several million people worldwide. The fundamental principal of retinal detachment repair is closure of the retinal break(s), or tamponade. F. W. Newell, Ophthalmology: Principles and Concepts, 6^{th} Ed., C. V. Mosby Co., St. Louis, 1986. Conventional means of tamponade consist of a) scleral buckling surgery (placement of a soft silicone band sewn to the external sclera to compress holes in the retina); b) primary placement of halogenated gases in the vitreous cavity via injection (known as pneumatic retinopexy); or c) placement of halogenated gases or silicone fluids as internal tamponades inside the vitreous cavity at the time of pars plana vitrectomy (removal of the vitreous gel), with or without a scleral buckle. Scleral buckling has not been adequate to close retinal holes in all patients, and conventional internal tamponades are less dense than the aqueous vitreous. The conventional internal tamponades float upward and therefore have been inadequate for treating inferior retinal holes, leaving large portions of the retina untreated.

Mechanism of Retinal Detachment

The posterior segment of the eye includes (from inside outwards) the vitreous gel, neurosensory retina, and choroid (heavily vascular). The retinal photoreceptors receive essential metabolic support from the retinal pigment epithelium (RPE).

Retinal detachment occurs when the retina separates from the RPE, resulting in eventual death of the retina and subsequent loss of vision. As a normal part of aging, the vitreous gel can undergo liquefaction, collapse and separation from the retina. Separation of the vitreous gel may result in formation of a tear in the retina at a site of vitreo-retinal adhesion. The retinal tear provides a pathway for vitreous fluid to pass through and underneath the retina, thus detaching the retina from the choroid.

The goal of surgery is to close the holes in the retina, preventing further fluid flow into the sub-retinal space, allowing for reattachment of the retina. F. W. Newell, supra; J. Gonin, Ann. D'Oculist (Paris) 132 (1904), p. 30; R. Y. Foos and N. C. Wheeler, “Vitreoretinaljuncture. Synchysis senilis and posterior vitreous detachment,” Ophthalmology, 89(12), 1502 (1982). Conventional techniques to treat retinal detachment are as follows. For example, a scleral buckle consisting of a crosslinked polydimethylsiloxane band may be sewn to the outside of the eye to compress the wall of the eye inward and close the holes in the retina.

Other conventional methods employ halogenated gas or polydimethylsiloxane (silicone) fluids as internal tamponades. G. G. Giordano, M. F. Refojo, “Silicone Oils as Vitreous Substitutes,” Progress in Polymer Science, 23, 509-532 (1998); L. Larsson and S. Osterlin, “Posterior vitreous detachment. A combined clinical and physiochemical study,” Graefes Arch. Clin. Exp. Ophthalmol., 223(2), 92-95 (1985); L. M. Spencer, R. Y. Foos and B. R. Straatsma, “Enclosed bays of the ora serrata. Relationship to retina tears,” Arch. Ophthalmol., 83(4), 421-425 (1970); C. L. Schepens and G. C. Bahn, Arch. Ophthalmol. 44, 677 (1950). These materials can be placed inside the vitreous cavity and block the hole in the retina. Conventionally used internal tamponades (SF₆, C₃F₈ or polydimethylsiloxane fluid) float up to force the retina against the choroid, but leave the inferior retina unprotected. Conventional scleral buckling involves suturing a soft, elastomeric silicone band to the equatorial sclera with moderate morbidity in every case, and with occasional severe complications including intraocular hemorrhage and loss of vision. Moreover, current internal tamponades fill the vitreous cavity, decreasing vision, and contact anterior chamber structures, contributing to the formation of cataracts and glaucoma. Foos et al., supra; Schepens et al., supra; E. Custodis, Ber Deutssche Ophthalmol. Ges., 57, 227 (1952).

Conventionally, a patient with uncomplicated superior retinal detachment (i.e., with a retinal break in the upper 6 clock hours) can be treated immediately in the doctor's office by intraocular injection of tamponade material under eyedrop anesthesia, usually in less than 20 minutes. The patient then needs to maintain strict positioning guidelines for several days to keep the retinal break closed while the RPE absorbs the sub-retinal fluid. If this is successful, the patient can undergo laser treatment to create a scar around the retinal break which keeps it closed.

A patient with uncomplicated inferior retinal detachment (i.e., with a retinal break in the lower 6 clock hours) typically must undergo scleral buckling surgery. If the macula is threatened or detached, then any delay of surgery can negatively influence his/her outcome. Conventional scleral buckling surgery is major surgery, performed in an operating room, and requires between one and two hours (not including pre-operative or anesthesia procedures), depending on the case and the surgeon. Retinal detachment is not considered a surgical emergency, so operating room administrators will not move other less urgent cases for a scleral buckle. That means the physician and the patient need to find the earliest available time for the procedure. There is evidence to show that doing these procedures at night with the on call staff results in poorer outcomes (P. R. Lichter and P. R. Lichter, “The Timing of Retinal Detachment Surgery: Patient and Physician Considerations,” Ophthalmology, 99(9), 1349-1350 (1992)), so the procedure is often delayed several days, and this can result in the macula detaching or remaining detached. Because of the inconvenience, intrinsic delays, increased risk, and poorer outcomes, conventional scleral buckling surgery has been generally unpopular among patients and surgeons alike.

Thus, a better approach is wanted than the conventional repair of retinal detachment, such as conventional suturing a semi-solid silicone band to the external scleral wall. It would be advantageous if the conventional process of suturing the traditional scleral buckle to achieve indentation, one of the riskiest parts of the conventional procedure which can occasionally produce destructive bleeding and loss of vision, could be avoided.

U.S. Pat. No. 6,749,844 (patented Jun. 15, 2004) (“Magnetic fluids”) by Riffle et al. discloses treating retinal detachment in an eye by applying a magnetized scleral buckle. There is disclosed a scleral buckle comprising a flexible biocompatible material, suitable for application to the sclera, preferably a flexible silicone band, dimensioned to fit snugly around the eye and gently compress the eye so that the inner surface of the vitreal chamber is urged into contact with the periphery of the retina. The magnetic scleral buckle is positioned generally by suture or adhesive.

U.S. patent application Ser. No. 10/620,762, published May 6, 2004 (U.S. Pat. Application No. 20040086572), for “Delivery of therapeutic agent affixed to magnetic particles,” by Dailey and Riffle, discloses certain therapeutic uses of magnetic particles injected into the eye.

More generally, a variety of magnetic systems for surgical, medical or other patient-related approaches have been disclosed, such as U.S. Pat. No. 5,125,888 issued Jun. 30, 1992 and U.S. Pat. No. 6,216,030 issued Apr. 10, 2001, both to Howard et al. for “Magnetic stereotactic system for treatment delivery;” U.S. Pat. No. 5,654,864 issued Aug. 5, 1997 to Ritter et al. for “Control method for magnetic stereotaxis system;” U.S. Pat. No. 5,707,335 issued Jan. 13, 1998 and U.S. Pat. No. 5,779,694 issued Jul. 14, 1998 both to Howard et al. for “Magnetic stereotactic system and treatment delivery;” U.S. Pat. No. 5,931,818 issued Aug. 3, 1999 to Werp et al. for “Method of and apparatus for intraparenchymal positioning of medical devices;” U.S. Pat. No. 6,015,414 issued Jan. 18, 2000 and U.S. Pat. No. 6,475,223 issued Nov. 5, 2002, both to Werp et al. for “Method and apparatus for magnetically controlling motion direction of a mechanically pushed catheter;” U.S. Pat. No. 6,157,853 issued Dec. 5, 2000, U.S. Pat. No. 6,212,419 issued Apr. 3, 2001, U.S. Pat. No. 6,304,768 issued Oct. 16, 2001, and U.S. Pat. No. 6,507,751 issued Jan. 14, 2003, all to Blume et al. for “Method and apparatus using shaped field of repositionable magnet to guide implant;” U.S. Pat. No. 6,241,671 issued Jun. 5, 2001 to Ritter et al. for “Open field system for magnetic surgery;” U.S. Pat. No. 6,292,678 issued Sep. 18, 2001 to Hall et al. for “Method of magnetically navigating medical devices with magnetic fields and gradients, and medical devices adapted therefor;” U.S. Pat. No. 6,311,082 issued Oct. 30, 2001 and U.S. Pat. No. 6,529,761 issued Mar. 4, 2003, both to Creighton et al. for “Digital magnetic system for magnetic surgery;” U.S. Pat. No. 6,330,467 issued Dec. 11, 2001 and U.S. Pat. No. 6,630,879 issued Oct. 7, 2003, both to Creighton et al. for “Efficient magnet system for magnetically-assisted surgery;” U.S. Pat. No. 6,459,924 issued Oct. 1, 2002 to Creighton et al. for “Articulated magnetic guidance systems and devices and methods for using same for magnetically-assisted surgery;” U.S. Pat. No. 6,505,062 issued Jan. 7, 2003 to Ritter et al. for “Method for locating magnetic implant by source field;” U.S. Pat. No. 6,522,909 issued Feb. 18, 2003 to Garibaldi et al. for “Method and apparatus for magnetically controlling catheters in body lumens and catheters;” U.S. Pat. No. 6,702,804 issued Mar. 9, 2004 and U.S. Pat. No. 6,755,816 issued Jun. 29, 2004, both to Ritter et al. for “Method for safely and efficiently navigating magnetic devices in the body;” U.S. Pat. No. 6,542,766 issued Apr. 1, 2003 to Hall et al., for “Medical devices adapted for magnetic navigation with magnetic fields and gradients.” Many of these mentioned patents are issued to Stereotaxis, Inc. Also, U.S. Pat. Pub. No. 20030135112 by Ritter et al. dated Jul. 17, 2003 for “Method of localizing medical devices;” U.S. Pat. Pub. No. 20010038683 by Ritter et al. dated Nov. 8, 2001 for “Open field system for magnetic surgery;” U.S. Pat. Pub. No. 20040030244 by Garibaldi et al. dated Feb. 12, 2004, for “Method and apparatus for magnetically controlling catheters in body lumens and cavities;” U.S. Pat. Pub. No. 20040096511 by Harbum et al. dated May 20, 2004 for “Magnetically guidable carriers and methods for the targeted delivery of substances in the body;” U.S. Pat. Pub. No. 20040199074 by Ritter et al. dated Oct. 7, 2004 for “Method for safely and efficiently navigating magnetic devices in the body;” U.S. Pat. Pub. No. 20040157082 by Ritter al. dated Aug. 12, 2004 for “Coated magnetically responsive particles, and embolic materials using coated magnetically responsive particles.”

Certain magnetic polymer particles, made by certain production processes, have been disclosed for certain biological/medical applications, see, e.g., U.S. Pat. No. 4,335,094 issued Jun. 15, 1982 to Mosbach for “Magnetic polymer particles;” U.S. Pat. No. 4,795,698 issued Jan. 3, 1989 to Owen et al. (Immunicon Corp.) for “Magnetic-polymer particles”; U.S. Pat. No. 5,814,687 issued Sep. 29, 1998 to Kasai et al. (JSR Corp.) for “Magnetic polymer particle and process for manufacturing the same”; U.S. Pat. No. 5,866,099 issued Feb. 2, 1999 to Owne et al. (Nycomed Imaging AS) for “Magnetic-polymer particles”.

### SUMMARY OF THE INVENTION

Constructing a fixed biomagnetic structure in living tissue by photo-initiated polymerization has been invented. This novel photo-initiated polymerization to construct, in living tissue, a polymer including ferromagnetic particles may be exploited in biomedical and surgical applications.

For example, biomagnetic manipulation in living tissue may be elegantly accomplished. An especially beneficial advantage is that the invasiveness of some conventional surgeries (such as retinal repair, for example) can be reduced, and biomagnetic components can be situated without suturing. Medical procedures (such as, e.g., repair of retinal detachment) conventionally needing suturing can now be accomplished without needing suturing. For example, in the case of repairing retinal detachment, one or more may be used of: a magnetized system (such as, e.g., a magnetized scleral buckle in conjunction with a magnetic fluid); and polymerization in situ (i.e., in the eye) of a polymer including ferromagnetic particles. In inventive retinal repair, an effective internal tamponade agent (such as, e.g., a silicon magnetic fluid) may be used to close the retinal break, avoiding the necessity for indentation of the sclera produced by a traditional scleral buckle. Further, by using the present invention, suturing the traditional scleral buckle to achieve indentation, a relatively risky aspect of conventional retinal repair procedures, may be avoided.

In one preferred embodiment, the invention provides a method of medical repair in living tissue, comprising: (a) polymerizing ferromagnetic particles in situ into a polymer; and (b) with the polymer containing the polymerized ferromagnetic particles, controlling placement of a magnetic fluid (such as, e.g., a silicone magnetic fluid) in situ, such as, e.g., medical repair methods wherein no suturing is performed, medical repair methods wherein retinal detachment is repaired; medical repair methods wherein the polymer containing the polymerized ferromagnetic particles forms a magnetic scleral buckle; medical repair methods including placing the magnetic scleral buckle with a blunt cannula; methods including photo-initiated polymerization; etc.

In another preferred embodiment, the present invention provides a method of repair of retinal detachment, wherein retinal detachment is repaired without needing suturing, comprising a step of placing a magnetized scleral buckle in situ; such as, e.g., methods wherein the magnetized scleral buckle is placed with a blunt cannula (such as, e.g., methods wherein the magnetic scleral buckle is injected in situ via a blunt cannula and the injection of the polymer causes local hydrodissection of Tenon's capsule (or conjunctiva), which remains in place everywhere else around the eye, thus holding the polymer in its intended place, with fibrosis then occurring around the polymer thus permanently fixing the polymer in place); methods wherein the magnetized scleral buckle comprises a polymer containing ferromagnetic particles; methods including generating, in situ, an internal tamponade; methods wherein the magnetized scleral buckle is a polymer containing ferromagnetic particles that polymerizes in situ; methods wherein the magnetized scleral buckle has a magnetic strength sufficient to hold a biocompatible magnetic fluid in a desired place; etc.

In yet a further embodiment, the present invention provides a magnetic system, comprising: at least a fixed magnetized structure (such as, e.g., a scleral buckle, etc.) including a polymer containing ferromagnetic particles (such as, e.g., Nb—Fe—B ferromagnetic particles; magnetite ferromagnetic particles; cobalt ferromagnetic particles; iron ferromagnetic particles; nickel ferromagnetic particles; etc.), wherein the fixed magnetized structure is biocompatible and is in living tissue; and a biocompatible magnetic fluid; such as, e.g., systems including a drug or therapeutic agent being delivered (such as, e.g., systems in which a drug or therapeutic agent is continually released for drug delivery); systems wherein toxin separation is effected; etc.

The invention in another preferred embodiment provides a magnetized scleral buckle, comprising a polymer containing ferromagnetic particles; such as, e.g., a scleral buckle wherein the ferromagnetic particles are magnetite particles; etc.

The invention also provides an embodiment that is a method of repairing an eye suffering from inferior retinal detachment, comprising: with a needle (preferably, a 27 gauge or smaller needle) injecting into the eye an amount of a silicone magnetic fluid; and, forming in the eye, without suturing, a magnetic scleral buckle; such as, e.g., methods wherein the injecting step and the scleral buckle forming step are performed in an office setting or other non-operating room setting; methods wherein the magnetic scleral buckle forms by being polymerized in the eye; methods wherein the silicone magnetic fluid and the magnetic scleral buckle establish a structure which repairs the retinal detachment; etc.

The invention in another preferred embodiment provides a method of manipulating living tissue, comprising: from magnetic particles and other polymer-forming material, polymerizing a magnetic structure in the living tissue; such as, e.g., methods including magnetically operating the polymerized magnetic structure in the living tissue; methods including a step of delivering a magnetic fluid into the living tissue; methods wherein the magnetic fluid is injected into the living tissue; methods including applying a magnetic field to the polymerized magnetic structure in the living tissue to move a region of the living tissue as desired; methods wherein the applied magnetic field is created within the living tissue; methods wherein a magnet outside the living tissue is applied; using a magnet (or magnetic polymer) to move magnetic nanoparticles (such as, e.g., nanoparticles bound to a drug/therapeutic agent, etc.) to a target area in the body; using a magnet or magnetic polymer to facilitate diffusion of materials (such as, e.g., materials bound to nanoparticles) across tissue (such as, e.g., sclera) (such as, e.g., methods to facilitate intraocular drug delivery without intraocular injection of a drug); using magnetic force to move materials across tissue; constructing an artificial sphincter in a patient, or repairing or strengthening a sphincter in a patient; ameliorating urinary incontinence; etc.

In a further embodiment, the invention provides a process of producing a magnetic polymer, comprising the step of polymerizing a magnetic polymer in vivo in a patient, such as, e.g., a process of producing a magnetic polymer, comprising the steps of: delivering a starting material comprising ferromagnetic particles into a patient (such as, e.g., delivering the starting material by injection); and polymerizing the starting material into a magnetic polymer in the patient (such as, e.g., polymerizing by photo-initiation); etc.

### BRIEF SUMMARY OF THE DRAWINGS

FIG. 1 is a schematic illustration of an inventive embodiment including a system of a silicone magnetic fluid 2 positioned inside an eye (E) in apposition to an external, permanently magnetic band (i.e., magnetized scleral buckle 1).

FIG. 2 shows a schematic approach to forming well-defined polymer-magnetite complexes for steric stabilization in PDMS carrier fluids, for use in the present invention. Each chain covers approximately 0.8 nm² of surface area on the magnetite surface.

FIG. 3 is a reaction scheme for synthesis of PDMS surfactants used to form complexes with magnetite nanoparticles.

FIG. 4 is a graph of response of Nb—Fe—B particles to a magnetic field.

FIG. 5 is a graph of decay of polar moment of Nb—Fe—B particles in silastic.

FIG. 6 is a transmission electron micrograph of PDMS-magnetite complexes dispersed in PDMS fluid. The particle diameter in FIG. 6 is 7.4±1.7 nm.

FIG. 7 is a magnetization curve for a PDMS-magnetite magnetic fluid for use in inventive eye surgery. The fluid composition is 50 wt % of a 15,000 g mol⁻¹ PDMS carrier fluid, 31 wt % magnetite (from elemental analysis), and 19 wt % of a 1400 g mol⁻¹ PDMS dispersion stabilizer (with 3 carboxylate groups on one end).

FIG. 8 is a diagram of quantification of cell toxicity of magnetite polysiloxane fluids in an MTT assay.

FIG. 9 is a graph of results of the MTT assays, suggesting that the magnetite-polydimethylsiloxane fluids are non-toxic to three different cell lines: 1) Prostate cancer C₄₋₂ cells, 2) Human retinal pigment epithelial cells (HRPE), and 3) ARPE cells.

FIGS. 10A, 10B are graphs for ERG analysis done on rabbits with intraocular silicone magnetic fluid in place for 1 month (OD experimental, OS control).

FIG. 11 is a representation of a catheter 11 useable in an inventive embodiment in which intravascular delivery of a drug is provided.

### DETAILED DESCRIPTION OF A PREFERRED EMBODIMENT OF THE INVENTION

Certain medical repairs in living tissue (such as repair of retinal detachment, etc.) now may be accomplished using a suture-free system, such as, e.g., a magnetically-based system, such as, e.g., by a biocompatible fixed magnetic structure (such as, e.g., a magnetized scleral buckle) used with a biocompatible magnetic fluid; a 360°, by a stable tamponade; etc. Avoidance of sutures is mentioned as an advantage, and not as a requirement for practicing the invention.

A fixed magnetic structure has been mentioned for use in the present invention. Preferably the fixed magnetic structure (such as a scleral buckle, etc.) to be used in the medical repair (such as, preferably, retinal repair) of the present invention is formed from a polymer, such as, e.g., preferably a crosslinked polymer, most preferably a photo-initiated crosslinked polymer.

Photo-initiated crosslinked polymers have been used successfully in medical applications for many years. (For instance, silica fillers have been dispersed into dimethacylate monomers to produce photo-polymerizable dental restorative materials.) Photo-initiated polymerizations offer several advantages over thermally initiated polymerizations for biomedical applications, by providing an efficient route to rapid polymerization at temperatures acceptable to the biological environment. In addition, such photo-initiated polymer materials can be placed into unique spatial arrangements allowing the polymer to be inserted into precise locations because the starting materials are liquids. The liquid state can also result in enhanced tissue adhesion due to physical interlocking with surfaces. Another advantage of using photo-initiated polymers is that only minimally invasive techniques are required for introducing the liquid monomers into tissue (i.e., a syringe can be used for precise placement between tissues and fiber optic cables can generate the light that provides initiation of the curing reaction).

A magnetic fluid can be manipulated using magnetic fields. An example of a biocompatible magnetic fluid to use in the invention is, e.g., a silicone magnetic fluid. A preferred example of a silicone magnetic fluid useable in the present invention is silicone magnetic fluids based on block copolymers bound to ferromagnetic nanoparticles, which complexes are finely dispersed in polydimethylsiloxane fluid. As dispersion stabilizers, block copolymers are more efficient than homopolymers. The “anchor” block of the stabilizer is designed to strongly adsorb onto the particle surface and the “tail” block(s) protrude into the medium to stabilize the nanoparticles against coalescence. Examples of polydimethylsiloxane magnetic fluids that may be used as the silicone magnetic fluid of the present invention include, e.g., polydimethylsiloxane magnetic fluids based on magnetite, polydimethylsiloxane magnetic fluids based on cobalt, etc. Polydimethylsiloxane magnetic fluids are mentioned as examples, and the inventive is not limited to polydimethylsilixonae magnetic fluids.

Magnetic fluids based on magnetite are a preferred example of magnetic fluids useable in the present invention. Magnetite is a known substance. Magnetite is an iron oxide with an inverse spinel crystalline structure and has the molecular formula Fe₃O₄ (FeO.Fe₂O₃). Half of the Fe⁺³ is tetrahedrally coordinated to oxygen and the remaining half of the Fe⁺³ and all of the Fe⁺² is octahedrally coordinated to oxygen.

In constructing magnetic fluids for use in the present invention, biocompatibility is required. It should be kept in mind that the most common ferromagnetic metals, nickel, iron and cobalt, have been reported to be toxic to biological structures in their zero-valent (unoxidized) states. For example, the disease process siderosis bulbi describes the damage done by (unoxidized) iron to epithelial structures within the eye. The most common mechanism for iron toxicity appears to be oxygen dependent iron-stimulated free radical reactions. B. Halliwell and J. M. Gutteridge, “Biologically relevant metal ion-dependent hydroxyl radical generation. An Update,” FEBS Lett., 307, 108-112 (1992). By contrast, magnetite particles are already used as MRI contrast agents, and have been well evaluated and found to be essentially non-toxic and biocompatible. R. Weissleder, D. D. Stark, B. L. Engelstad, B. R. Bacon, C. C. Compton, D. L. White, P. Jacobs and J. Lewis, “Superparamagnetic iron oxide: pharmacokinetics and toxicity,” Am J. Roentgenol, 152(1), 167-173 (1989).

Carboxylic acid functional groups bind strongly to magnetite. B. Berkovsky and V. Bashtovoy, eds., Magnetic Fluids and Applications Handbook, Begell, N.Y., 1996. Thus, a an approach to preparing magnetic nanoparticles for dispersion into biocompatible polydimethylsiloxane carrier fluids is to: a) prepare a polydimethylsiloxane (PDMS) surfactant with appropriate binding groups, b) bind the new surfactant to magnetite nanoparticle surfaces, then c) disperse these into well-defined PDMS fluids.

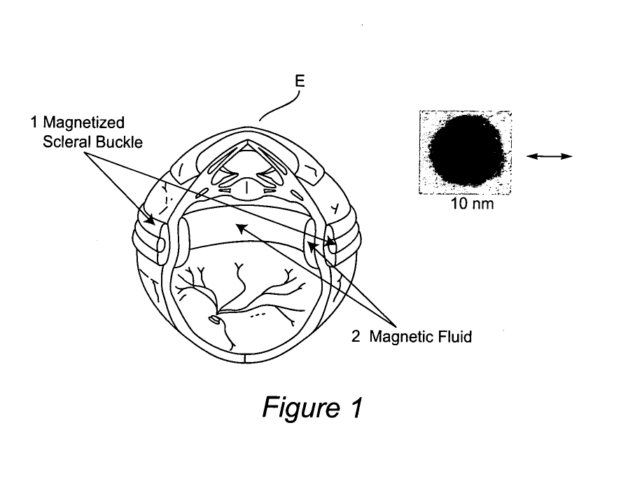

Referring to FIG. 1, in an inventive embodiment, a magnetized scleral buckle 1 is used in cooperation with a magnetic fluid 2 for repairing retinal detachment in an eye (E). The magnetic fluid 2 is biocompatible, such as, e.g., a silicone magnetic fluid. The magnetic fluid may be injected into the eye (E) with a needle (preferably, a very small needle (e.g., a 23 ga. needle, 25 ga. needle, 27 ga. needle, etc., preferably a 27 ga. needle)), without vitrectomy (a major operation in which the vitreous gel is removed). The magnetized scleral buckle 1, rather than being pre-constructed outside the eye (E) and delivered to the eye (E) by suturing or adhesive as in conventional procedures may be formed (such as by, e.g., polymerization) in the eye. For example, the starting materials from which to effect the polymerization of the magnetized scleral buckle 1 may be delivered to the eye (E) by a blunt cannula. Thus, inferior retinal detachments which conventionally have needed to be repaired in the operating room now can be repaired in the office.

Injecting the magnetic scleral buckle 1 in situ via a blunt cannula causes local hydrodissection of Tenon's capsule (or conjunctiva), which remains in place everywhere else around the eye (E), thus holding the polymer in its intended place. Then fibrosis will occur around the polymer (as fibrosis occurs around conventionally used scleral buckling material). The fibrosis will permanently fix the polymer in place.

With regard to the magnetic fluid (such as magnetic fluid 2 in FIG. 1) used in inventive medical repair (such as retinal repair in FIG. 1), there may be used to advantage a non-Newtonian nature of the magnetic fluid, in which non-uniform dispersion is exhibited, which correspondingly means that shear forces go to nearly zero on the edge of a needle, which in turn means that a retinal repair procedure using such a magnetic fluid may be office-based. A retinal repair procedure that can be performed in an office or other non-operating room setting is an important advance, in that cost and morbidity can be decreased, and by allowing for the procedure to be done in a more timely fashion.

Another advantage that the present invention provides for retinal repair is mentioned as follows. The eye has protective layers on the outside. On the back, the Tenon's capsule is thick and multi-layered, adherent but not continuous. If the smooth tip of a relatively blunt cannula is used to slice back and insert into the eye, when the polymer is inserted, hydrodissection occurs and the rest of the Tenon's is disinclined to move unless force is exerted. The fact that the Tenon's will stay in place in such a manner is advantageous, because the polymer is thereby held in place. Local inflammation and a capsule is formed. A buckle effect (which in conventional scleral buckling surgery closes the retinal break) is hence not necessary because the magnetic fluid (held in place over the retinal break by the outside polymer) closes the retinal break.

Such advantages and preferred details are mentioned for inventive retinal repair. However, polymerized magnetic materials may also be used in the eye and in other living tissue for other medical repairs. Retinal repair has been prominently mentioned for using the present invention, however, the invention is not limited thereto. A magnetically-cooperating system which exploits polymerization of a magnetic polymer in living tissue additionally may be used in a variety of biomedical applications, such as, e.g., drug delivery systems, toxin separations, repair of retinal detachment, repair of intracranial aneurisms by occlusion, medical imaging, etc.

For example, a highly viscous, biocompatible fluid containing suspended superparamagnetic particles with aligned magnetic moments may be placed into a tissue layer via syringe. A magnetized elastomer may thereby be formed. This magnetized elastomer may then be photo-crosslinked to form a biocompatible, fixed magnetic structure within layers of tissue.

As mentioned, a 360°, stable tamponade for treating retinal detachment has been invented. For example, a magnetic polydimethylsiloxane nanoparticle fluid may be placed inside the vitreous cavity, and a magnetic exoplant may be inserted in the potential space between the sclera and Tenon's capsule. The magnetic exoplant holds the ferrofluid securely at a retinal break in direct apposition to the exoplant. The central vitreous cavity (and visual axis) will be free of the magnetic fluid, and without contact between the magnetic fluid and the lens, anterior chamber structures, or macula. Complications of conventionally available treatment modalities for retinal detachment may thus be avoided.

As the silicone magnetic fluid for use in the present invention may be used, e.g., poly(dimethylsiloxane)-nanomagnetite complexes and dispersions in polysiloxane carrier fluids, and other silicone magnetic fluids known in the art (see, e.g., J. P. Dailey, J. P. Phillips, C. Li, and J. S. Riffle, “Synthesis of Silicone Magnetic Fluids for Use in Eye Surgery,” J. of Magnetism and Magnetic Materials, Apr. 1, 1999, 140-148; K. S. Wilson, M. Rutnakornpituk, L. A. Harris and J. S. Riffle, “Silicone magnetic fluids using poly(dimethylsiloxane)-b-poly(2-ethyl-2-oxazoline) as a steric stabilizer,” Polym. Prepr., 43(1), 732-733 (2002); Treatment with Magnetic Fluids, U.S. Pat. No. 6,135,118 to J. P. Dailey, Oct. 24, 2000; Magnetic Fluids, U.S. Pat. No. 6,464,968 to J. S. Riffle, J. P. Phillips and J. P. Dailey, VA Tech Intellectual Properties, Oct. 15, 2002; etc.), and other silicone magnetic fluids.

Using a silicone magnetic fluid may provide improvements compared to conventional retinal repair in at least the following areas: quality of tamponade; patient positioning; surgical complications; cost; re-operation; post-operative refractive error—anisometropia. An inventive approach to retinal repair may comprise a three hundred sixty degree internal tamponade system to treat both primary and complicated retina detachment.

Working in living tissue (such as, e.g., retinal repair) has been mentioned for certain inventive embodiments. When using the present invention in working in living tissue (such as retinal repair in a human eye), a sufficient magnetic field to accomplish the desired repair or such is wanted (such as, e.g., for operating a magnetic scleral buckle to accomplish retinal repair). On the other hand, when working in living tissue, excessive magnetic field strength inconsistent with work in living tissue should be avoided. Magnetic field strength may be controlled by controlling the ferromagnetic particle content in the fluid and in the polymer. For example, a level of magnetic strength may be ascertained by considering a commercially available fixed magnet, whose seller reports its magnetization. For example, a 4 mm by 8 mm by 2 mm Nd—Fe—B magnet commercially available from MagnaQuench provides 3,000 to 3,200 gauss magnetic strength at its surface, 884 gauss magnetic strength at about 3 mm, and 265 gauss magnetic strength at about 6 mm. Such a magnet causes a fluid of nanoparticles to brisly exit a syringe needle tip and travel about 1 cm, which is significantly more magnetic strength than needed to accomplish retinal repair.

With regard to other applications of the present invention, when establishing a magnetic field for use in a living tissue according to the present invention, the strength of the magnetic field preferably should be in a range that accomplishes a desired operation without being harmful (or at least is only minimally harmful) to the living tissue. Strength of the magnetic field may be manipulated for different applications by selecting bulk magnetization of the ferromagnetic material chosen, ferromagnetic particle size and distance.

The present invention may advantageously used, in certain embodiments, for moving a certain region of living tissue, in a relatively non-invasive manner, by polymerizing (such as by photo-initiated polymerization) a magnetic structure in a desired region of living tissue, and then by applying a magnetic field to move the polymerized structure as desired. For example, a polymer on one side of a tissue may be used for moving nanoparticles to the other side of the tissue. In another example, a polymer on the posterior surface of the outside of the eye may be used to cause nanoparticles injected inside the eye to collect on the posterior surface of the inside of the eye.

When a magnetic field is applied to a polymerized magnetic structure (such as a magnetic structure that has been polymerized in situ in living tissue), the magnetic field being applied to the polymerized magnetic structure may be from something magnetically cooperating within the living tissue (such as, e.g., a magnetic fluid, preferably, a biocompatible magnetic fluid such as, e.g., a silicone magnetic fluid, etc.), or from something magnetic outside the living tissue (such as a traditional magnet, etc.).

In each application, a particular shape of a fixed magnetic structure is wanted, such as, e.g., a shape of a scleral buckle (FIG. 1) for retinal repair. The invention is not particularly limited to a scleral buckle shape, and encompasses all medically useful shapes and pharmaceutically useful shapes which may be polymerized as fixed magnetic structures in living tissue.

Other customized shapes of fixed magnetic structures may be designed for particular surgical applications, pharmaceutical applications, etc., such as, e.g., fixed magnetic supporting structures, fixed magnetic structures inclusive of a drug, fixed magnetic structures relating to drug delivery but not themselves including a drug; etc. For constructing a desired shape of a fixed magnetic structure, ferromagnetic particles and other materials from which to form a polymer may be delivered to a region in living tissue, and, when the ferromagnetic particles and other polymer-forming materials are in place, polymerizing conditions may be carried out, such as photo-initiated polymerization, to form the desired fixed magnetic structure.

The following inventive Examples mentioned, but it will be appreciated that the invention is not limited to the Examples.

### EXAMPLE 1

Magnetic Nb—Fe—B microparticles were placed into a medium of Silastic A (Dow-Corning) and magnetic properties were measured via SQUID magnetometry.

Preparation of a Dispersion of Nb—Fe—B Microparticles in Silastic-A:

A 5-mL vial was charged with 2.9954 g of Silastic A 9280-50 and 0.9200 g of Nb—Fe—B microparticles (MagneQuench, Inc.). The mixture was stirred with a stainless steel spatula and sonicated for 5 minutes at 50% power. The resulting material was a highly viscous dispersed mixture that showed little signs of settling after several days of observation.

Measurement of Response of Nb—Fe—B Particles to an Applied Magnetic Field

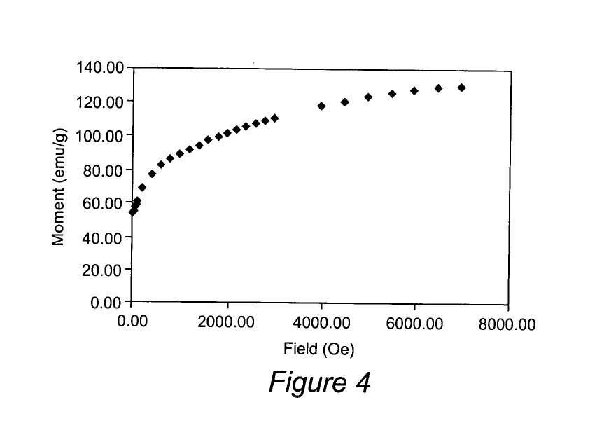

A 15-mg sample of dry Nb—Fe—B particles was measured at room temperature in response to applied magnetic fields. Measurements were made from 0 to 70 kOe at 25° C. While the magnetic field was insufficient to saturate the particles, FIG. 4 demonstrates that the slope of the magnetic response decreased substantially as the field was increased. At 70 kOe the particles had a moment of 128.12 emu/g.

Measurement of the Decay of Magnetic Moment with Time

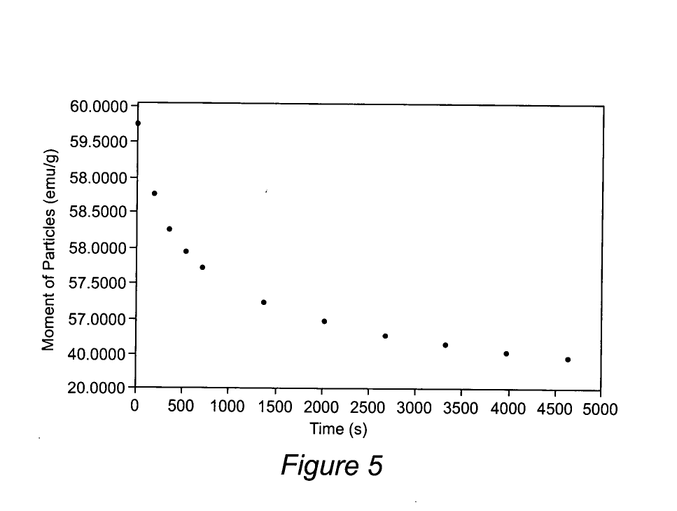

A 42.4-mg sample of the Silastic A/particle dispersion was studied via SQUID. The sample was placed in a 70,000 Oe field to magnetize the sample, then the field was turned off. The magnetic moment of the sample was measured with time at zero field. After 15 minutes the magnetic moment of the suspension decreased to 57.20 emu/g. Very little further decay was observed over 80 minutes. These data are plotted in FIG. 5. The ability to maintain such a moment for this extended time suggests the feasibility of creating photo-initiated, crosslinked magnetic networks in tissues.

The above experimentation of this Example 1 as seen with reference to FIGS. 4 and 5 demonstrates that microscale particles can be suspended in a crosslinkable polysiloxane medium. The resultant dispersion was sufficiently viscous to maintain the aligned magnetic moments of the particles long enough to allow for insertion into the tissue and for photo-polymerization. The data in this Example 1 demonstrate feasibility of, for example, a magnetized scleral buckle. The data give further appreciation for the usefulness of producing a unique polymer to maximize the binding efficiency of microparticles in a crosslinkable polymer medium.

### EXAMPLE 2

### Preparation of Magnetite Nanoparticles

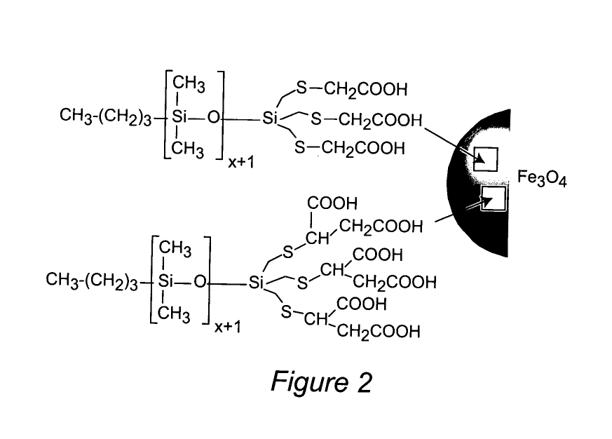

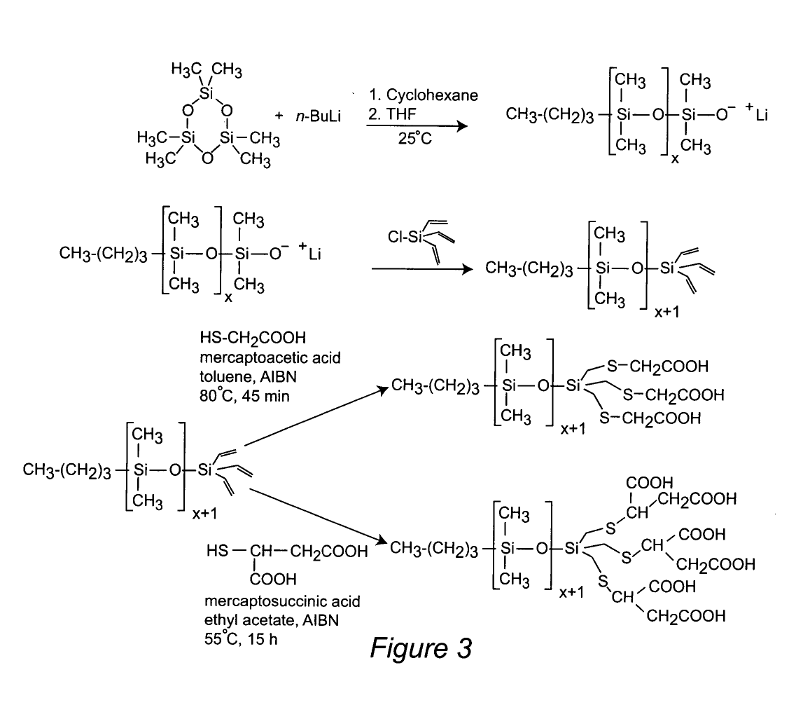

Carboxylic acid-functionalized PDMS surfactants were synthesized for steric stabilization of magnetite nanoparticle dispersions in biocompatible polysiloxane carrier fluids (FIG. 2). Trivinylsilyl-terminated PDMS was prepared via living polymerization of D₃, then reacted with either mercaptoacetic acid or mercaptosuccinic acid using a free radical thiol-ene addition to afford PDMS containing either three or six carboxylic acid groups at one end (FIG. 3). Magnetite nanoparticles were prepared by chemically co-precipitating FeCl₂ and FeCl₃ at pH 9-10, then the PDMS-magnetite nanoparticle complexes were prepared via interfacial adsorption of the carboxylate groups of the PDMS stabilizer onto aqueous magnetite particles at a slightly acidic pH.

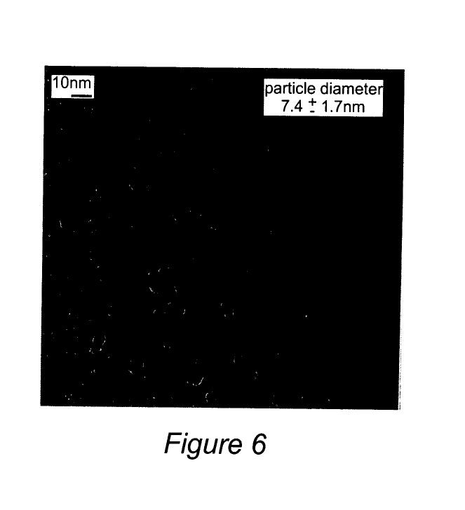

Repeated centrifugations to remove any aggregates resulted in well-dispersed polymer-magnetite nanoparticle complexes. The complexes were characterized with transmission electron microscopy to establish an average particle diameter of 7.4±S.D. 1.7 nm and approximately spherical shape (FIG. 6). The compositions of the polymer-magnetite nanoparticle complexes were analyzed to understand the amount of surface space that each carboxylate group covered (≈0.2 nm² unless one sterically places the carboxylic acid groups too close). This now provides a means for predicting the amount of surfactant needed for optimum particle coverage and to enable good dispersion in the PDMS carrier fluids.

Vibrating sample magnetometry was utilized to determine the magnetic responses of the optimized complexes. Complexes containing up to 67 wt % magnetite were prepared with these PDMS dispersion stabilizers, resulting in complexes with saturation magnetizations of ˜50 emu g⁻¹. The complexes were dispersed into polysiloxane carrier fluids by ultrasonication, resulting in magnetically responsive polysiloxane fluids.

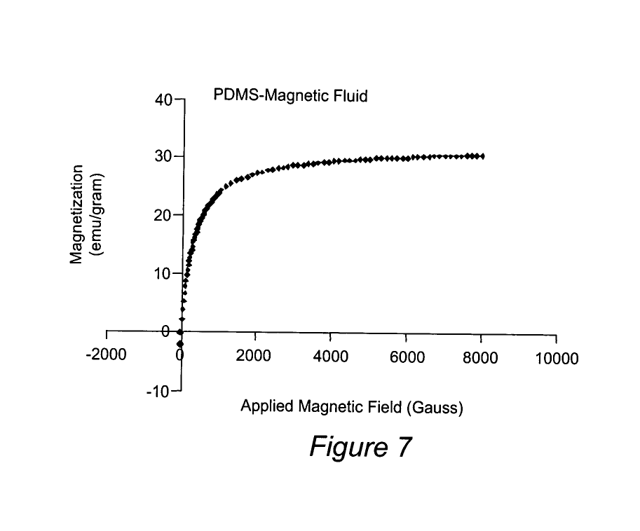

The magnetization curve illustrated in FIG. 7 shows the behavior of these fluids as a function of applied field strength. Magnetization curves show the response of the fluids as field is increased, then as the field is decreased. The steep rise in magnetization at low fields in FIG. 7 notes high magnetic susceptibility (good response at low applied fields). The response as field is decreased exactly overlays the response as field is increased (so that only one curve is visible). This signifies that when the applied field is removed, the vector magnetic moments of the dispersed magnetite nanoparticles randomize quickly (losing their magnetic response, i.e., they have no memory). This property is known as superparamagnetic behavior, and is typical of such small nanoparticles. Fluid dispersions of small magnetic nanoparticles respond to applied magnetic field gradients by moving as a whole fluid toward the direction of highest field. Thus, when these fluids are placed near a permanent magnet, the entire fluid body flows toward the magnet as an entity.

### EXAMPLE 3

### In Vitro Evaluations of Magnetite Silicone Magnetic Fluid

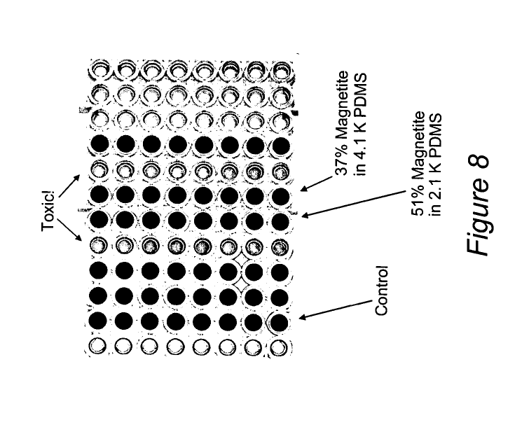

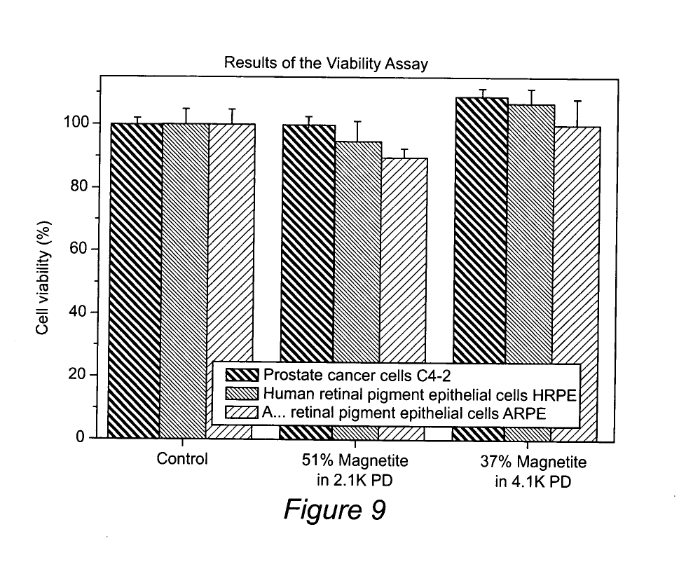

In-vitro evaluations of the magnetite silicone magnetic fluid of Example 2 have been conducted using the well-established MTT Assay in which the tetrazolium salt MTT (3-(4,5-dimethylthiazol-2-yl)-2,5-diphenyl-tetrazolium bromide) is enzymatically reduced in living, metabolically-active cells but not in dead cells. The reactions were carried out in multi-well plates, and the purple formazan reaction product was disssolved in dimethylsulfoxide, and measured by visible spectroscopy (FIG. 8). Referring to FIG. 8, magnetic fluids or their supernatants were incubated with human retinal pigment epithelial cells (HRPE) or C₄₋₂ prostate cancer cells for 48 to 72 hours. Viability was then measured in a 96-well plate. Healthy cells oxidize the yellow dye MTT into a blue formazan product, which is then quantified at 540 nm in a well plate reader. The assay results (FIG. 9) suggest that the magnetite-polydimethylsiloxane fluids were not toxic to any of the cell lines investigated.

### EXAMPLE 3A

With confocal microscopy, the HRPE cells which had been cultured on slide surfaces coated with the silicone magnetic fluid were examined. Healthy growth of the HRPE cells on the slide surfaces was observed with no cases of entry of the magnetic fluid into the cells. The healthy growth of RPE cells in the presence of the silicone magnetic fluid suggests strongly that the fluid does not inhibit cell growth.

### EXAMPLE 3B

The magnetophoretic behavior of the magnetite silicone magnetic fluid was observed in a cow cadaver eye. A 4×8×2 mm NdFeB magnet was sutured to the external sclera. The cornea, iris, lens, and vitreous gel were removed, and the vitreous cavity was filled with balanced salt solution. The magnetic silicone fluid was injected via a 20 ga. cannula into the mid-vitreous. The fluid moved directly and briskly toward the magnet (actually toward the retinal surface opposite the magnet). It formed a single layered body along the surface of the retina opposite the magnet. We observed no magnetic fluid anywhere else in the eye. Moreover, the eye was vigorously shaken, and the magnetic fluid remained an intact body and did not diffuse into the balanced salt solution.

### EXAMPLE 3C

Four rabbits were each injected with 0.15 mL of the silicone magnetic fluid into the vitreous cavities of their right eyes. The left eyes served as controls. The animals were examined at one day, one week, and one month with indirect ophthalmoscopy. At one month, electroretinography was performed, and the animals underwent fundus photography. The animals were sacrificed and the eyes were processed by standard technique for light microscopy. Where possible, sections were taken from the areas of the extrascleral magnets.

Extensive sectioning of the retinas was performed. Histology of the retinas in the pilot investigation showed no significant differences between the experimental and control eyes. The retinal toxicity described in the literature is not subtle. For example, with perfluoro-n-octane, Chang et al. reported that “photoreceptor outer segments were distorted, the outer plexiform layer was narrowed, and pre-retinal accumulation of macrophages had occurred.” S. Chang, J. R. Sparrow, T. Iwamoto, A. Gershbein, R. Ross and R. Ortiz, “Experimental Studies of Tolerance to Intravitreal Perfluoro-n-octane Liquid,” Retina, 11(4), 367-374 (1991). ERG's were normal in the control and experimental eyes.

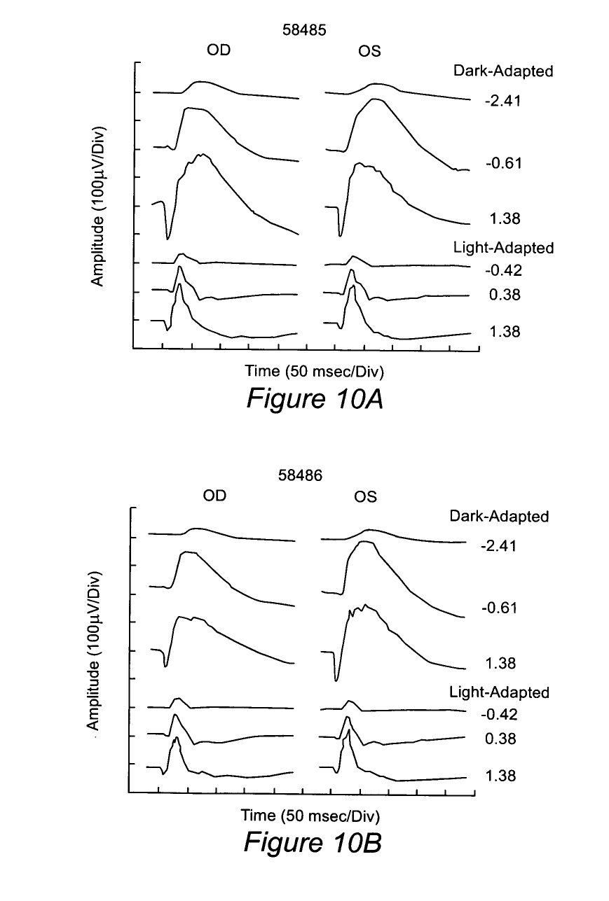

With light microscopic evaluation and of all of our sections, and ERG analysis of the subjects, there was no evidence of toxicity (FIGS. 10A, 10B). Also, it is notable that a) we were able to inject the magnetic fluid easily via 27 ga. needle (please see “Viscosity” below), b) without performing pars plana vitrectomy (i.e., through the intact vitreous body of young rabbits), and that c) the magnetic fluid moved directly and briskly in the direction of the external magnet with d) good apposition to the retina throughout the entire period of the experiment, and e) no emulsification throughout the entire time of the experiment (1 month).

### EXAMPLE 3D

### Stability of Magnetic Silicone Fluids In Vivo

The magnetic silicone fluid of Example 3 was carefully observed for the one-month term of the animal experiments of Examples 3B, 3C to qualitatively evaluate its stability against emulsification or any changes in dispersion quality (note that these are two separate issues). The multi-functional (carboxylate-functional) polydimethylsiloxane dispersion stabilizers in the magnetic fluid are strongly bound to the magnetite particle surfaces as a result of their multi-functionality and the molecular spacing between carboxylate functional groups. The macromolecular polydimethylsiloxane “tails” of these dispersants extend into the silicone carrier fluid to maintain steric (entropic) separation between magnetite nanoparticles so that they do not aggregate. The fluid dispersion remained intact throughout the period and there was no evidence of any changes in the dispersion quality. Macromolecular silicone dispersants strongly bound to the magnetic nanoparticles impart stability against aggregation due to steric repulsion.

In literature reports of conventional silicone oil, emulsification occurs in 85-100% of cases at 6 months. J. L. Federman and H. D. Schubert, “Complications Associated with Silicone Oil in 150 Eyes after Retina-Vitreous Surgery,” Ophthalmology, 95, 870 (1988). By contrast, the magnetic materials studied in this Example 2D appear to resist emulsification. These inventive fluids differ from conventional (non-magnetic) silicone fluids in that magnetic forces bind these fluids together, and this may be related to their durability against emulsification. It is also important to note that it is the migration of emulsified conventional silicone oil droplets that is implicated in complications including keratopathy and glaucoma. The magnetic fluid is held closely in place by the extrascleral magnet. In experiments with NdFeB extrascleral magnets, that force was easily sufficient to draw the fluid briskly to the magnet from anywhere in the eye. Thus, even if some emulsification had occurred, the droplets would still be unlikely to escape.

### EXAMPLE 3E

### Viscosity of the Silicone Magnetic Fluid

The silicone oil conventionally in clinical use is a simple (Newtonian) fluid. By contrast, the magnetic fluid of this inventive Example 3E is a dispersion of nanoparticles in a carrier medium and, as such, is a complex (or non-Newtonian) fluid. Shear force does little to change the viscosity of Newtonian silicone fluids. They remain viscous during injection and removal. Thus, the conventional fluids must be injected (after pars plana vitrectomy) through a relatively large cannula (20 ga.), and this usually requires assistance of a mechanical pump. Removal of conventional silicone oil also requires a trip to the operating room and exchange with fluid or gas. By contrast, shear force dramatically reduces the viscosity of the silicone magnetic fluid, and this causes it to become much less viscous during passage through a small gauge needle. It was unexpectedly found, surprisingly, that the magnetic fluid of this inventive example can easily be injected via a 27 ga. needle. Moreover, with the external magnet in place, it also passes immediately through the formed vitreous of the rabbit subjects and sits against the retina. This is important for several reasons. Firstly, intraocular injection of silicone magnetic fluid (with sub-Tenon's injection of the proposed magnetic paste) may be considered as an office procedure using only topical anesthesia. Secondly, where removal of the magnetic fluid from the eye might be wanted or needed, topical anesthesia extraction of the magnetic fluid and removal of the sub-Tenon's polymer is shown to be realistic.

The incidence of retinal detachment in the U.S. is greater than 1 in 10,000 individuals per year. Approximately 10% of all retinal detachments are complicated by proliferative vitreo-retinopathy, and approximately 40% include inferior retinal breaks. The above experimental data of Examples 1-3E indicate that a system of silicone magnetic fluid and magnetic scleral buckling may be safe and effective for treating retinal detachment in such individuals. Thus, between 2,000 and 10,000 individuals (hurnans) in the United States each year may be able to benefit from inventive silicone magnetic fluid and magnetic scleral buckling systems.

### EXAMPLE 4

### Retinal Repair

Silicone magnetic fluid is injected into the vitreous cavity. The magnetic fluid is held in place by a soft magnetic silicone network inserted into the sub-Tenon space directly opposite the retinal break.

Insertion of a soft magnetic magnet to hold the magnetic silicone fluid tamponade securely at a site in apposition to a retinal break. A patient with uncomplicated retinal detachment with retinal break(s) in the inferior 6 clock hours undergoes the following. After pupillary dilation, the patient is examined with scleral depression and the meridian(s) of the retinal break(s) is localized with a sterile marker. Eyedrop anesthesia (topical tetracaine) is applied. With Westcott scissors, 1-mm incisions are made into the sub-Tenon space near the conjunctival formix. A curved sub-Tenon cannula is passed through the incision(s) under indirect ophthalmoscopic visualization. The illuminated cannula serves to identify the location of the retinal break in relation to the external sclera (at the cannula tip). When the cannula tip is adjacent to the retinal break, a magnetic silicone paste is injected from the cannula and polymerized (crosslinked) in-situ by the cannula's illumination (through a visible light initiated reaction similar to that currently used in dentistry). The 1-mm trans-conjunctival incisions near the conjunctival formix will not require suturing.

Insertion of the magnetic silicone fluid tamponade. The surgeon injects 0.05-0.1 mL of the silicone magnetic fluid in the meridian of the previously-marked retinal break(s), via a 27-gauge needle, 3.5 mm from the corneo-scleral limbus. Topical medication including antibiotic, steroid, and atropine are applied, and the patient is sent home. The patient is instructed to avoid strenuous activity, to sleep in any position that is comfortable, and to return the next day for follow-up examination. In a follow-up visit when the sub-retinal fluid has been absorbed, laser treatment is applied to the perimeter of the retinal break(s).

In the case of complicated retinal detachment (e.g., proliferative vitreoretinopathy), which occurs in about 10% of retinal detachment patients, the patient undergoes vitrectomy and membrane dissection as is conventionally done, but instead of a conventional scleral buckle, an inventive magnetic scleral buckle (see FIG. 1) or external magnetic paste is used, depending on the surgeon's preference. Instead of using conventional internal tamponades (e.g., silicone fluid, sulfur hexafluoride or perfluoropropane gases), silicone magnetic fluid is used.

A silicone magnetic fluid as an internal tamponade for treating retinal detachment may provide one or more of the following advantages.

Quality of tamponade. Conventionally, there have been no reliable means of tamponading inferior retinal breaks. The inventive system of magnetic fluid and magnetic paste or network provides a stable internal tamponade at virtually any site on the retina that the surgeon chooses.

Positioning. Conventional post-operative positioning required makes surgical repairs non-feasible in many cases (the elderly, orthopedic, pulmonary, cardiac problems, injury). The inventive approach makes post-operative positioning unnecessary.

Reducing complications. Complications associated with conventional scleral buckling surgery include: a) Hemorrhage. Macular hemorrhage resulting from drainage of sub-retinal fluid occurs in up to 10% of cases and usually results in permanent visual reduction. Suprachoroidal hemorrhage is associated with placement of scleral buckle sutures (especially posterior ones) and can result in catastrophic loss of vision; b) Intra and extraocular inflammation and infection. Orbital inflammation occurs more or less in all cases of scleral buckling surgery. Choroidal effusion is common. Bacterial orbital cellulites and endophthalmitis occur infrequently with scleral buckling surgery. Complications of conventional internal tamponade include: a) Glaucoma and cataract. The leading complications of conventional silicone oil tamponades result from contact with anterior chamber structures. With the magnetic fluid in the present inventio, there need not be contact with anterior chamber structures; b) Post-op pressure elevations occur as a result of the surgeon's need to obtain a complete silicone oil or gas fill. Complete fill is not necessary with the inventive approach.

Simplification of primary surgery. The present invention may be performed as office-based procedures. By contrast, conventional scleral buckling surgery requires an operating room, anesthesiologist, anesthesia materials, etc.

Re-operation. For conventional procedures, re-operation after placement of conventional silicone oil is required in nearly every case (conventional silicone is present in the visual axis and contributes to cost and complications including glaucoma, corneal decompensation and cataract). With the inventive approach, re-operation is occasionally necessary.

Anisometropia—refractive shifts. Conventional scleral buckles close retinal breaks by significantly indenting the scleral wall and as a result nearly always produce refractive shifts and anisometropia, often resulting in chronic vision problems in successful cases. Silicone magnetic fluid with a magnetic scleral buckle according to the present invention close the retinal break by the internal tamponade effect of the silicone magnetic fluid, held in place by the magnetic scleral buckle, so indentation would usually not be required (although indentation of the magnetic scleral buckle may be occasionally required to reduce vitreoretinal traction). So in most cases anisometropia and refractive shifts would not be an issue with the silicone magnetic fluid.

### EXAMPLE 5

### Drug Delivery

Macular degeneration is a leading cause of blindness. Delivering a drug (such as a drug similar to an aptomer) to scar tissue that causes the blindness is wanted. The role of vascular endophilial growth factor (VEGF) in macular degeneration has been studied. Anti-VGEF actomers have been widely studied in clinical trials. Conventionally, anti-VGEF drugs are injected into the eyeball.

This treatment may be improved by creating a non-magnetic polymer that holds and slowly releases the anti-VGEF drug, allowing diffusion across the sclera. Thus, release over a period of years, as opposed to a period of about 2-3 weeks as with conventional injection, may be targeted by a an unassisted diffusion method.

Additionally, an assisted diffusion method may be provided, in which the anti-VGEF drug is attached to ferromagnetic particles and a magnetic field is used to move the anti-VGEF drug across the sclera. The present invention may be applied in such a method.

In an application of the present invention, the anti-VGEF drug may be delivered to the eye by injection or by diffusion, and then be collected using a magnetic field (e.g., a magnetic polymer) in the back of the eye. Thus, the drug may be caused to collect in the back of the eye where its delivery is wanted. As scar tissue occupies only about five percent of the region in this disease, the drug could be concentrated on that affected area of scar tissue, with less drug being delivered and affected healthy areas.

### EXAMPLE 6

### Treating Aneurism by Intracranial Occlusion

Intracranial aneurisms due to vascular malformations in the brain are seen in some patients, usually appearing at age 20 or after. An early conventional approach to repairing such malformations was first by surgical clipping or chopping, which required dissection to reach the aneurism. The dissection was problematic because some neurological function was invariably lost.

Rogers Ritter and others subsequently proposed an approach using a catheter to place a catheter which could inject polymer up into the central nervous system (CNS) vasculature.

According to this inventive Example 6, the catheter is magnetically guided to the site of an intracranial aneurysm and polymer is injected into the sac of the aneurysm and polymerized in situ, which leads to a clot, and fibrosis and scarring. Thus, by promoting and manipulating scarring, the aneurism may be treated by intracranial occlusion.

### EXAMPLE 7

### Gastrointestinal Imaging

Conventionally, gastro intestinal imaging may be accomplished by a patient swallowing a huge pellet that is almost egg-sized and thus relatively difficult to swallow.

The present invention may improve upon such technology by substituting a liquid for the patient to swallow in place of a solid that is conventionally swallowed. For example, a liquid solution that will be polymerizable into a magnetic polymer in situ in a patient (such as in a patient's digestive system) may be swallowed by a patient.

A magnetic polymer so swallowed in liquid form may be caused, while in the patient's digestive system, to polymerize in a circumstance for an imaging application, such as, e.g., as a contrast agent for gastro intestinal studies.

### EXAMPLE 8

### Concentration of Pro-Angiogenic Factors

Diabetes patients have problems with peripheral circulation and wound healing. Limbs are often lost because of poor blood flow and poor wound and fracture healing.

The present invention may be applied to ameliorate such problems. Magnetic drug delivery may be used in such patients with wound or fracture healing to concentrate pro-angiogenic factors. Blood flow may thereby be improved. For example, such a magnetic drug delivery application may be used in orthopaedic surgery, and other contexts. Concentration of pro-angiogenic factors using magnetic drug delivery may also be used in peripheral neuropathy, neurology applications, etc.

### EXAMPLE 9

### Intravascular Drug Delivery

Intravascular delivery of drugs has been identified as valuable to attempt but has faced difficulties in achieving success. Guiding something to be delivered through the bloodstream poses problems of the huge forces encountered. For example, blood travels at about 3 m/sec from the descending aorta. Also, huge turbulence is present.

When values are established for a size of a material to be delivered, and a speed at which the material is traveling, the applicable magnetic force equation may then be considered. An example of a size of material that might be designed for intravascular delivery is an inner diameter of about 1 to 2 mm (which would be considered relatively big). An example of a speed at which a material might be delivered is about 10 to 20 cm/sec (which would be considered relatively fast). With those values, the applicable force equation may be considered. The magnetic force exerted on such a particle would be affected by bulk magnetism of the material, the diameter of the particle, the distance that the particle is from the magnet, and the rate at which the particle is moving.

When a drug-carrying particle is traveling in the bloodstream, there is the problem of how to make the particle select as wanted at a Y intersection that it encounters. For effective drug delivery, the drug-containing particle must be made to turn where the designer wants it to turn.

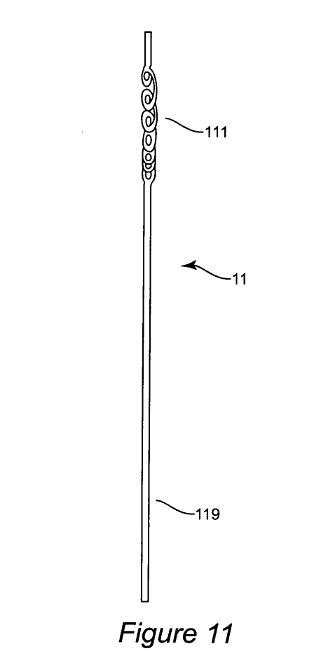

The present inventors have established that with a magnet of approximately 8,000 gauss magnetic strength at a distance of 1.5 cm from a particle moving at 5 cm/sec, the moving particle can be made to turn as desired at a Y intersection. On the basis of this observation, it was determined to slow the flow. A helical-shaped catheter (such as, e.g., a catheter 11 as shown in FIG. 11) may be used to inject drug-containing particles and to slow the flow. With reference to FIG. 11, the catheter 11 includes a winding helical area 111 and a non-helical area 119. The diameter of the helical area 111 is about the same as the catheter 11 and the diameter of the catheter within the helical area 111 is about the same as the rest of the catheter 11. The catheter 11 is hollow, like a drinking straw. The catheter 11 is inserted and positioned in the blood stream and may be guided by an external magnetic field (such as Stereotaxis, Inc.'s external magnetic field). Ferromagnetic nanoparticles carrying a drug are sent through the catheter 11.

In terms of the point to which the flow may be slowed, others have extensively studied the rate of blood flow at which clotting will, and will not, occur. The inventive intravascular drug delivery including slowing of blood flow is to be operated within those established parameters relating to clotting, so that clotting is avoided.

Steering of the ferromagnetic nanoparticles carrying the drug is performed via use of a magnetic field.

### EXAMPLE 10

### Biological Anchoring

The present invention may be used to develop and improve upon conventional magnetic anchoring devices that have been disclosed for biological use, such as, e.g., those disclosed by Gannoe et al., in U.S. Pat. No. 6,656,194 (patented Dec. 2, 2003) (“Magnetic anchoring devices”), in which devices are inserted into the stomach of a patient and certain magnetic coupling was effected.

While the invention has been described in terms of its preferred embodiments, those skilled in the art will recognize that the invention can be practiced with modification within the spirit and scope of the appended claims.

1. A method of medical repair in living tissue, comprising:
polymerizing ferromagnetic particles in situ into a polymer; and
with the polymer containing the polymerized ferromagnetic particles, controlling placement of a magnetic fluid in situ.

2. The method of claim 1, wherein the magnetic fluid is a silicone magnetic fluid.

3. The medical repair method of claim 1, wherein no suturing is performed.

4. The medical repair method of claim 1, wherein retinal detachment is repaired.

5. The medical repair method of claim 1, wherein the polymer containing the polymerized ferromagnetic particles forms a magnetic scleral buckle.

6. The medical repair method of claim 5, including placing the magnetic scleral buckle with a blunt cannula.

7. The method of claim 1, including photo-initiated polymerization.

8. A method of repair of retinal detachment, wherein retinal detachment is repaired without needing suturing, comprising a step of placing a magnetized scleral buckle in situ.

9. The method of claim 8, wherein the magnetized scleral buckle is placed with a blunt cannula.

10. The method of claim 8, wherein the magnetized scleral buckle comprises a polymer containing ferromagnetic particles.

11. The method of claim 8, including generating, in situ, an internal tamponade.

12. The method of claim 8, wherein the magnetized scleral buckle is a polymer containing ferromagnetic particles that polymerizes in situ.

13. The method of claim 12, wherein the magnetized scleral buckle has a magnetic strength sufficient to hold a biocompatible magnetic fluid in a desired place.

14. A magnetic system, comprising:
at least a fixed magnetized structure including a polymer containing ferromagnetic particles, wherein the fixed magnetized structure is biocompatible and is in living tissue; and
a biocompatible magnetic fluid.

15. The magnetic system of claim 14, wherein the fixed magnetized structure is a scleral buckle.

16. The magnetic system of claim 14, including a drug being delivered.

17. The magnetic system of claim 14, wherein toxin separation is effected.

18. The magnetic system of claim 14, including magnetic particles selected from the group consisting of Nb—Fe—B; magnetite; cobalt; iron and nickel.

19. A magnetized scleral buckle, comprising a polymer containing ferromagnetic particles.

20. The scleral buckle of claim 19, wherein the ferromagnetic particles are magnetite particles.

21. A method of repairing an eye suffering from inferior retinal detachment, comprising:
with a needle injecting into the eye an amount of a silicone magnetic fluid;
forming in the eye, without suturing, a magnetic scleral buckle.

22. The method of claim 21, wherein the injecting step and the scleral buckle forming step are performed in an office setting or other non-operating room setting.

23. The method of claim 21, wherein the magnetic scleral buckle forms by being polymerized in the eye.

24. The method of claim 21, wherein the silicone magnetic fluid and the magnetic scleral buckle establish a structure which repairs the retinal detachment.

25. A method of manipulating living tissue, comprising:
from magnetic particles and other polymer-forming material, polymerizing a magnetic structure in the living tissue.

26. The method of claim 25, including magnetically operating the polymerized magnetic structure in the living tissue.

27. The method of claim 26, including a step of delivering a magnetic fluid into the living tissue.

28. The method of claim 27, wherein the magnetic fluid is injected into the living tissue.

29. The method of claim 25, including applying a magnetic field to the polymerized magnetic structure in the living tissue to move a region of the living tissue as desired.

30. The method of claim 29, wherein the applied magnetic field is created within the living tissue.

31. The method of claim 29, wherein a magnet outside the living tissue is applied.

32. A process of producing a magnetic polymer, comprising the step of:
polymerizing a magnetic polymer in vivo in a patient.

33. The process of claim 32, wherein the process includes delivering a starting material comprising ferromagnetic particles into the patient and polymerizing the starting material into a magnetic polymer in the patient.

34. The process of claim 33, wherein the step of delivering the starting material is by injection.

35. The process of claim 32, wherein the polymerizing step is by photo-initiation.
